(19) United States
(12) Patent Application Publication
(10) Pub. No.: US 2021/0369652 A1
(43) Pub. Date: Dec. 2, 2021

(54) Nonhormonal Unisex Contraceptives

(71) Applicant: The Regents of the University of California, Oakland, CA (US)

(72) Inventors: Polina V. Lishko, Berkeley, CA (US); William Skinner, Berkeley, CA (US); Liliya Gabelev Khasin, Berkeley, CA (US); Emiliano Tabarsi, Berkeley, CA (US); Ambre M. Bertholet, San Francisco, CA (US); Yuriy Kirichok, San Francisco, CA (US)

(73) Assignee: The Regents of the University of California, Oakland, CA (US)

(21) Appl. No.: 17/391,026

(22) Filed: Aug. 1, 2021

Related U.S. Application Data
(63) Continuation of application No. PCT/US20/19373, filed on Feb. 21, 2020.
(60) Provisional application No. 62/808,861, filed on Feb. 22, 2019.

(51) Int. Cl. A61K 31/167; A61K 31/166; A61K 31/4985; A61P 15/16; A61P 15/18

(52) U.S. Cl. CPC A61K 31/167; A61K 31/166; A61P 15/18; A61P 15/16; A61K 31/4985

(57) ABSTRACT

Nonhormonal unisex contraceptive products, compositions, formulations and methods of use comprise an effective amount of a targeted mild mitochondria uncoupler.

### CROSS-REFERENCE TO RELATED APPLICATIONS

This application claims the priority benefit of U.S. Provisional Application No. 62/808,861, filed Feb. 22, 2019, the contents of which are incorporated herein in their entirety.

### GOVERNMENT SUPPORT

This invention was made with government support under Grant Numbers GM111802, GM118939, and HD081403 awarded by the National Institutes of Health. The government has certain rights in the invention.

### INCORPORATION OF THE SEQUENCE LISTING

The contents of the file named B19-097_ST25.txt, which was created on Feb. 20 2020, and is 11 KB in size are hereby incorporated by reference in their entirety.

### BACKGROUND

The urgent need for contraception worldwide concerns billions of people. According to the National Health Statistics Report, 62 percent of women of reproductive age that use contraception are relying mostly on hormonal pills and intrauterine devices (IUDs)²¹. However, numerous reports indicate that despite being highly effective, undesirable side effects associated with hormonal contraceptives often result in discontinuation of their use^{6,8,9}. Steroid hormones are associated with powerful side effects, such as depression, weight gains, ectopic pregnancy and others because of their pleiotropic mode of action on various cell types. Almost a third of American women discontinue using hormonal contraceptives within the first year of use, due to the side effects. Additionally, the only modern options for male contraception are condoms, which have a high real-world failure rate, and vasectomies, which are surgically invasive. Therefore, there is a large unmet need for novel non-hormonal unisex contraceptives. Such a contraceptive could potentially be used by billions of people across the globe.

### SUMMARY

The disclosure demonstrates that targeted mild mitochondrial uncouplers, DNP, niclosamide, and BAM15, uncouple sperm mitochondria. Niclosamide, the most potent of the compounds, also decreased sperm beat frequency from 14 Hz to 6 Hz (FIG. 6A), inhibited hyperactivation (FIG. 6C) at concentrations as low as 1 μM.

The disclosure provides nonhormonal unisex contraceptive products, compositions, formulations and methods of use, which comprise an effective amount of a targeted mild mitochondria uncoupler.

The disclosure provides a method of promoting contraception, comprising administering to a person in need thereof a composition comprising an effective amount of a targeted mild mitochondria uncoupler.

In some embodiments of the methods of the disclosure, the uncoupler comprises or is a salicylanilide or salt thereof, such as an ethanolamine salt thereof, such as niclosamide ethanolamine (NEN)

In some embodiments of the methods of the disclosure, the uncoupler comprises or is a salicylanilide compound or salt thereof, and the salicylanilide compound is selected from: niclosamide, bromochlorosalicylanilide, oxyclozanide, rafoxanide, 3-tert-butyl-5-chloro-N-(2-chloro-4-nitrophenyl)-2-hydroxy-6-methylbenzamide, dibromsalan, metabromsalan, tribromsalan, and 2-iodo-N-phenylbenzamide (benodanil).

In some embodiments of the methods of the disclosure, the uncoupler comprises or is a salicylanilide compound or salt thereof, and the salicylanilide compound has a structure:

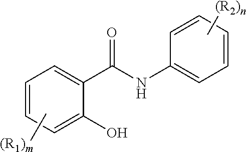

wherein R₁ and R₂ are independently halide, such as F, Cl, Br or I, substituted heteroatom selected from O and N, such as —OH, —NO₂ or O-Ph-Cl, or lower (C1-C4) alkyl, such as Me, Et, or t-butyl, m is an integer 1, 2, 3 or 4, and n is an integer 1, 2, 3, 4 or 5.

In some embodiments of the methods of the disclosure, the uncoupler comprises or is BAM15 (N5,N6-bis(2-Fluorophenyl)-[1,2,5]oxadiazolo[3,4-b]pyrazine-5,6-diamine), or salt thereof.

In some embodiments of the methods of the disclosure, the composition is administered via an oral route, a topical route, a rectal route, or a vaginal route.

In some embodiments of the methods of the disclosure, the composition is administered as a pill, a cream, a vaginal ring, a vaginal film or a patch.

In some embodiments of the methods of the disclosure, the composition is administered orally and the composition is administered as a pill

In some embodiments of the methods of the disclosure, the composition is administered topically and the composition is administered as a cream or a patch.

In some embodiments of the methods of the disclosure, the composition is administered intravaginally and the composition is administered as a cream, a patch, a vaginal ring or a vaginal film.

In some embodiments of the methods of the disclosure, the composition is administered intrarectally and the composition is administered as a cream or a patch.

The disclosure provides a use of a targeted mild mitochondria uncoupler of the disclosure as a contraceptive.

In some embodiments of the uses of the disclosure, the uncoupler comprises or is a salicylanilide or salt thereof, such as an ethanolamine salt thereof, such as niclosamide ethanolamine (NEN).

In some embodiments of the uses of the disclosure, the uncoupler comprises or is a salicylanilide compound or salt thereof, and the salicylanilide compound is selected from: niclosamide, bromochlorosalicylanilide, oxyclozanide, rafoxanide, 3-tert-butyl-5-chloro-N-(2-chloro-4-nitrophenyl)-2-hydroxy-6-methylbenzamide, dibromsalan, metabromsalan, tribromsalan, and 2-iodo-N-phenylbenzamide (benodanil).

In some embodiments of the uses of the disclosure, the uncoupler comprises or is a salicylanilide compound or salt thereof, and the salicylanilide compound has a structure:

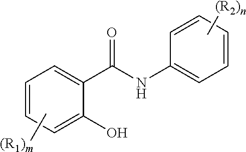

wherein R1 and R2 are independently halide, such as F, Cl, Br or I, substituted heteroatom selected from 0 and N, such as —OH, —NO2 or O-Ph-Cl, or lower (C1-C4) alkyl, such as Me, Et, or t-butyl, m is an integer 1, 2, 3 or 4, and n is an integer 1, 2, 3, 4 or 5.

In some embodiments of the uses of the disclosure, the uncoupler comprises or is BAM15 (N5,N6-bis(2-Fluorophenyl)-[1,2,5]oxadiazolo[3,4-b]pyrazine-5,6-diamine), or salt thereof.

In some embodiments of the uses of the disclosure, the composition is administered via an oral route, a topical route, a rectal route, or a vaginal route.

In some embodiments of the uses of the disclosure, thee composition is administered as a pill, a cream, a vaginal ring, a vaginal film or a patch.

In some embodiments of the uses of the disclosure, the composition is administered orally and wherein the composition is administered as a pill.

In some embodiments of the uses of the disclosure, the composition is administered topically and wherein the composition is administered as a cream or a patch.

In some embodiments of the uses of the disclosure, the composition is administered intravaginally and wherein the composition is administered as a cream, a patch, a vaginal ring or a vaginal film.

In some embodiments of the uses of the disclosure, the composition is administered intrarectally and wherein the composition is administered as a cream or a patch.

The disclosure provides a contraceptive formulation comprising a targeted mild mitochondria uncoupler. In some embodiments, the contraceptive formulation comprises an effective amount of the targeted mild mitochondria uncoupler. In some embodiments, the contraceptive formulation is in the form of a pill, capsule, suppository, cream, vaginal ring, vaginal film or patch.

The disclosure encompasses all combinations of the particular embodiments of the disclosure.

### BRIEF DESCRIPTION OF THE DRAWINGS

FIGS. 1A-C is a series of schematic diagrams showing that the sperm cell relies on its mitochondria to generate energy. (A) General diagram of energy conversion within mitochondria. (B) The mechanism of mitochondrial ATP and heat production. Mitochondria have two membranes, the outer mitochondrial membrane (OMM, freely permeable to ions and small molecules <1500 Da) and the IMM (which has much more tightly controlled permeability and contains the apparatus for ATP and heat production). The electron transport chain (ETC) generates potential (ΔΨ) across the IMM, which is used by ATP synthase (AS) to produce ATP and H⁺ leak pathways to generate heat. Modified from¹. (C) Spermatozoa rely on energy produced by their mitochondria that are tightly packed in the midpiece region of the tail. A proton gradient is established by the ETC to power the synthesis of ATP. In active swimming sperm cells, cellular ATP consumption causes active APT/ADP exchange via ANT that inhibits the ANT-mediated H⁺ leak, and ΔΨ is used to produce ATP only: (#1). When cellular activity is reduced, less ATP is consumed, leading to increases in ΔΨ and reactive oxygen species (ROS) production: (#2)^{2,3}. To prevent this from happening, proton leak through ANT increases, because now it is not inhibited by active ADP/ATP exchange (#3). This proton leak converts ΔΨ into heat, returning ΔΨ back to the normal value and decreasing ROS production. However, when ΔΨ is reduced by the proton leak, which is carried in sperm by ANT2 and ANT4, the mitochondria dissipate the energy in the form of heat and cannot produce enough ATP to power sperm motility¹².

FIG. 2 is a series of chemical structures of mild mitochondrial uncouplers: Niclosamide, BAM15 and 2,4-dinitrophenol.

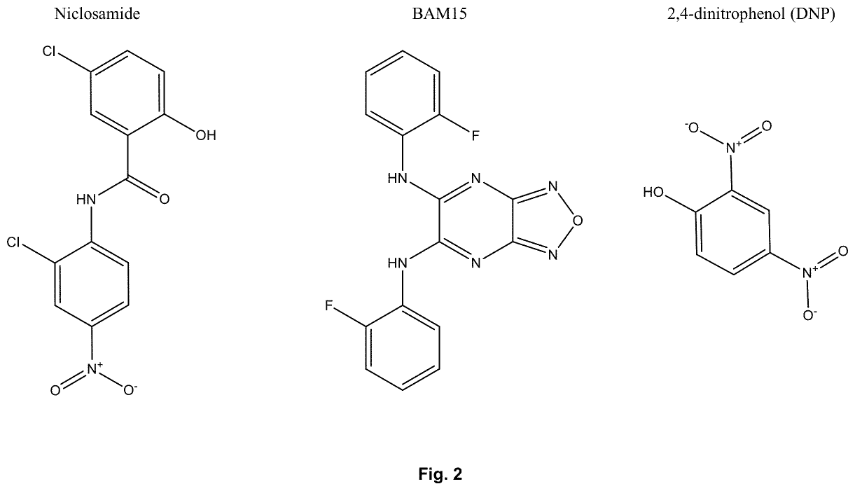

FIG. 3A-B is a pair of schematic diagrams depicting a mitochondrial patch-clamp. (A) Preparation of mitoplasts (vesicles of the whole IMM): (1) Isolation of mitochondria from cell lysate by centrifugation; (2) OMM removal using a low-pressure French press; (3) isolated mitoplasts in a KCl solution. Remnants of the OMM (arrow) are attached to the IMM. (B) Patch-clamp recording from mitoplasts. After formation of the gigaohm seal (mitoplast-attached configuration), the membrane patch under the pipette can be destroyed by high-amplitude voltage pulses to form a whole-mitoplast configuration for recording currents (I) across the whole IMM. Alternatively, the patch pipette can be withdrawn from the mitoplast to form an inside-out mode for recording single-channel activity.

FIG. 4 is a series of graphs (upper panels) and a schematic diagram (lower panel) showing regulators of ANT-mediated H⁺ current (I_{H}). Left panel: I_{H} activated by 2 μM arachidonic acid (AA) followed by a transient inhibition by 1 mM of bath ADP (2), and subsequent recovery (3). Control current is shown in black. Middle panel: I_{H} time course of the left panel. Skeletal muscle mitoplast. I_{H} amplitudes were measured upon stepping from 0 to −160 mV. Right panel: ADP inhibition of I_{H} in points 2 and 3. Remaining I_{H} measured at −160 mV is shown as a percentage of control, n=8. Explanation of transient I_{H} inhibition by cytosolic adenine nucleotides. ANT in c-state, with fatty acid (FA) anion in the translocation pathway, mediates I_{H} (I). Cytosolic ADP³⁻ binds in c-state and expels FA anion/blocks translocation pathway, leading to I_{H} inhibition (2). Upon ANT conformation change, ADP dissociates into matrix (pipette) solution (2 and 3). FA anion re-associates with ANT in m-state, restoring I_{H} (4 and 5). Cytosolic ADP cannot inhibit I_{H} while AAC is in m-state (5). Preliminary data from (Bertholet, A. M. Chouchani., E. T.; Kazak, L.; Angelin, A.; Fedorenko, A.; Long, J. Z.; Vidoni, S.; Garrity, R.; Cho, J.; Terada, N.; Wallace, D.C.; Spiegelman, B. M.; and Kirichok, Y. Proton Transport is an Integral Function of the Mitochondrial ADP/ATP Carrier Nature July; 571(7766):515-520 (2019)).

FIGS. 5A-B is a series of graphs demonstrating that ANT is required for H⁺ leak activated by FA and DNP. a, Representative currents induced by 2 μWI AA in WT and ANT1^{-/-} mitoplasts of heart. Right panel: I_{H} current densities at −160 mV for WT and ANT1^{-/-} mitoplasts. b, Representative currents induced by 50 μM DNP in WT and ANT1^{-/-} mitoplasts of heart. Right panel: I_{H} current densities induced by 50 and 200 μM DNP at −160 mV in WT and ANT1^{-/-} mitoplasts.

FIGS. 6A-C is a series of graphs demonstrating the impact of mitochondrial uncouplers (MU) on sperm physiology and fertilizing capacity. Niclosamide significantly decreases sperm motility, uncouples mitochondria and prevents fertilization. A) Human sperm were purified by swim-up method and their basal beat frequency (BF, in Hz) was measured as described in (Mannowetz, N., Naidoo, N. M., Choo, S. A., Smith, J. F. & Lishko, P. V. Slol is the principal potassium channel of human spermatozoa. eLife 2, e01009, doi:10.7554/eLife.01009 (2013)) in control HS solution ((1); in the presence of vehicle control ((2); DMSO), and in the presence of MU: 1 μM Niclosamide ethanolamine (NEN), 0.76 μM BAM15, and 5 μM DNP. Data are standard boxplots, with middle line representing median, upper and lower bounds of the box representing first and third quartiles, and whiskers representing maximum and minimum values, not including outliers. X's represent the mean of the sample, and dots represent individual data points. B) Human sperm mitochondrial membrane potential (MMP) assessment. Human sperm were incubated for 15 minutes in the presence of 0.1% DMSO (vehicle control), in the presence of 1 μM NEN or 0.75 μM BAM15. Samples from each condition (at least 100 of sperm midpieces per condition) were then assessed for fluorescence intensity measured with 45 nM MitoRed, which is a cell membrane permeable rhodamine-based dye. MitoRed localizes to mitochondria, and emits red fluorescence. The interaction of Mito Red with mitochondria depends on the membrane potential of the mitochondria. Uncoupled mitochondria emit less fluorescence. Data are standard boxplots, as in (A). C) Effect of sMU on human capacitated sperm hyperactivated motility. Cells were capacitated as described (11) for 4 hours. 4 μM Progesterone was added 2 minutes before imaging to trigger hyperactivation (HA). Cells were assessed as either Hyperactivated or Not Hyperactivated. N>45 cells. Error bars represent 95% CI calculated through the Wilson/Brown method.

### DETAILED DESCRIPTION

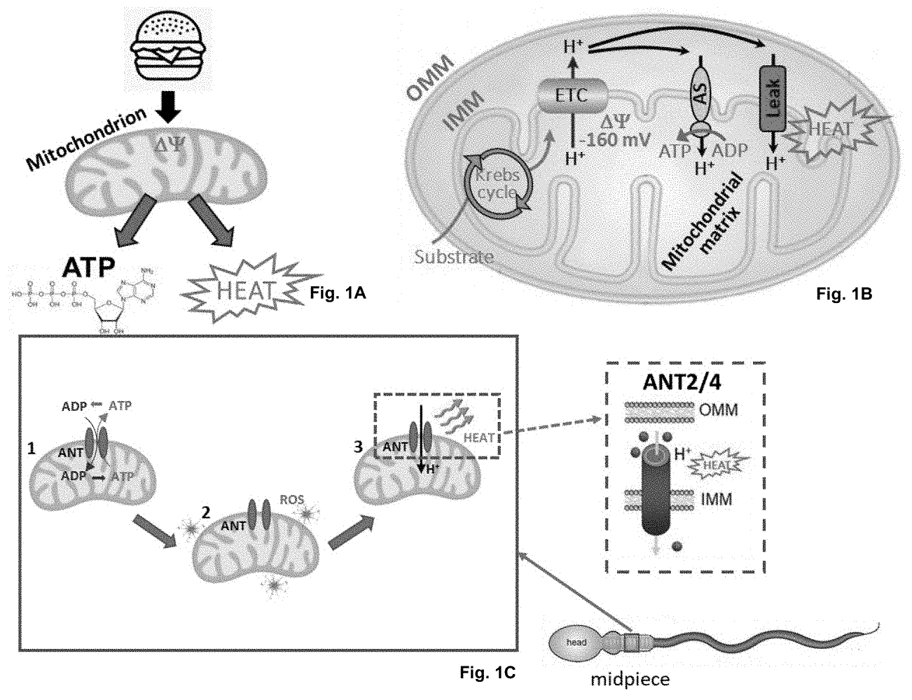

Mitochondria generate ATP by coupling the H⁺ transport activities of the mitochondrial electron transport chain (ETC) and ATP synthase²², two gigantic transport protein complexes located in the inner mitochondrial membrane (IMM). Specifically, the ETC, fueled by high-energy electron donors provided by the Krebs cycle, pumps H⁺ out of the mitochondrial matrix to generate an electrochemical H⁺ gradient (ΔΨ) across the IMM. ATP synthase then returns H⁺ back into the mitochondrial matrix down the ΔΨ and uses the released energy to synthesize ATP from ADP and inorganic phosphate. To maximize the energy transfer between ETC and ATP synthase, the chemiosmotic theory postulated that the IMM conductance for H⁺ (and other ions) must be close to zero. However, it is now well-established that the IMM of any tissue is “leaky” for H. The H⁺ leak (I_{H}) across the IMM is mediated by uncoupling proteins (UCPs). Similar to ATP synthase, UCPs return H⁺ back into the mitochondrial matrix down the ΔΨ, but do not generate ATP and instead dissipate the released energy as heat. This phenomenon, known as mitochondrial uncoupling, is crucial for mitochondrial function and integrity (FIG. 1). Free fatty acids (FA) are physiological activators of H⁺ leak via UCPs. Mitochondrial uncoupling in the specialized thermogenic tissues, brown and beige fat, helps to maintain core body temperature and control body weight^{3,23-25}, and is happening via uncoupling protein 1 (UCP1) that is brown and beige fat-specific. In contrast to brown fat, mitochondria of regular somatic and reproductive tissues are only “mildly” uncoupled, which implies a smaller I_{H}. However, because this mild uncoupling occurs in the majority of tissues, it may have a significant impact on thermogenesis, body weight, healthy metabolism and reproduction potential^{3,26}.

In addition, the mild uncoupling reduces mitochondrial reactive oxygen species (ROS) production to preserve mitochondrial integrity¹². The uncontrolled escape of electrons from the mitochondrial ETC to oxygen is the primary source of ROS in cells. The mild mitochondrial uncoupling that slightly reduces the potential across the IMM is a major mechanism that prevents ROS generation by ETC. Indeed, mild uncoupling was shown to be potentiated/activated by FA, ROS, and by hyperpolarized ΔΨ^{12,27}. Despite the importance of mild mitochondrial uncoupling, the molecular identity of UCP(s) in all tissues except for brown fat remained elusive.

Therefore, chemical protonophores, such as 2,4-dinitrophenol (DNP), have been widely used to induce I_{H} and mitochondrial uncoupling for research and therapeutic purposes. DNP is hydrophobic, membrane soluble weak acids that can carry H⁺ across biological membranes without the help of membrane transport proteins. What distinguishes them from FA (also hydrophobic weak acids, but poor protonophores) is the ability to diffuse through the membrane not only in the protonated form but also in the H⁺-free, negatively charged form. In accordance with the classic model, DNP, in the protonated form, carry H⁺ through the lipid bilayer to release it on the opposite side. Then, in the anionic form, they diffuse back across the membrane, bind another H⁺, and repeat the cycle. DNP was used to demonstrate that ΔΨ is essential for mitochondrial ATP production^{22,28}, providing key evidence for the chemiosmotic theory. Later, DNP was shown to increase body energy expenditure and thermogenesis, while dramatically reducing fat deposition and body weight²⁹. However, DNP has significant side effects in humans, and because it was considered a simple chemical protonophore without a protein target, efficiency and safety of DNP could not be improved²⁹.

However, recent reports challenge the classic dogma that known mitochondrial uncouplers such as DNP act as a membrane protonophore^{2,30}. These data strongly demonstrate that mild mitochondrial uncouplers induce the H⁺ leak by primarily interacting with Adenosine Nucleotide Transporter (ANT) proteins.

In humans and mice, the ANT protein has several isoforms that have a tissue-specific expression patterns with ANT4 being specifically expressed in the testis and sperm cells while completely repressed in other organs. It has been shown that ANT4-deficient male mice exhibit impaired spermatogenesis and are completely infertile¹³. It is important to note that these mice are viable and exhibit otherwise normal development and physiology¹³. ANT4 was previously proposed as a contraceptive target, and a drug screen¹⁴ was executed to look for compounds that inhibit ATP/ADP exchanger activity of ANT4; however, the lead compounds were found nonspecific as they have also inhibited other human ANT isoforms and therefore exhibited broad cytotoxicity.

The disclosure focuses on a completely different ANT transport modality—the ability to conduct protons and hence uncouple mitochondria in response to certain pharmacological intervention. Mild mitochondrial uncoupling is now recognized as a way to achieve weight loss, treat diabetes and even as an anticancer therapy¹⁵. Therefore, this ANT function is not expected to impose the cytotoxic effect on the targeted cells, but rather decrease their efficiency of ATP production.

In some embodiments of the disclosure, an ANT4 protein may comprise or consist of the amino acid sequence of ADP/APT translocase 4 (ANT4) (UniProtKB-Q9H0C2; also known as solute carrier family 25 member 31 (SLC25A31); transcript variant 1):

(SEQ ID NO: 1)

| | |
|---|---|
| 1 | MHREPAKKKA EKRLFDASSF GKDLLAGGVA AAVSKTAVAP |
| | IERVKLLLQV QASSKQISPE |
| 61 | ARYKGMVDCL VRIPREQGFF SFWRGNLANV IRYFPTQALN |
| | FAFKDKYKQL FMSGVNKEKQ |
| 121 | FWRWFLANLA SGGAAGATSL CVVYPLDFAR TRLGVDIGKG |
| | PEERQFKGLG DCIMKIAKSD |
| 181 | GIAGLYQGFG VSVQGIIVYR ASYFGAYDTV KGLLPKPKKT |
| | PFLVSFFIAQ VVTTCSGILS |
| 241 | YPFDTVRRRM MMQSGEAKRQ YKGTLDCFVK IYQHEGISSF |
| | FRGAFSNVLR GTGGALVLVL |
| 301 | YDKIKEFFHI DIGGR; |

or be encoded by the nucleic acid comprising or consisting of the sequence of:

(SEQ ID NO: 2)

| | |
|---|---|
| 1 | gcagcttttc cgcacgcgcc tcgccggcgc gcggctctct |
| | cagcgtccca agagccactt |
| 61 | tctcgccagt acgatgctgc agcggttttc cggttttccg |
| | cttcccttca tcgtagctcc |
| 121 | cgtactcatt tttagccact gctgccggtt tttatatcct |
| | tctccatcat gcatcgtgag |
| 181 | cctgcgaaaa agaaggcaga aaagcggctg tttgacgcct |
| | catccttcgg gaaggacctt |
| 241 | ctggccggcg gagtcgcggc agctgtgtcc aagacagcgg |
| | tggcgcccat cgagcgggtg |
| 301 | aagctgctgc tgcaggtgca ggcgtcgtcg aagcagatca |
| | gccccgaggc gcggtacaaa |
| 361 | ggcatggtgg actgcctggt gcggattcct cgcgagcagg |
| | gtttcttcag tttttggcgt |
| 421 | ggcaatttgg caaatgttat tcggtatttt ccaacacaag |
| | ctctaaactt tgcttttaag |
| 481 | gacaaataca agcagctatt catgtctgga gttaataaag |
| | aaaaacagtt ctggaggtgg |
| 541 | tttttggcaa acctggcttc tggtggagct gctggggcaa |
| | catccttatg tgtagtatat |
| 601 | cctctagatt ttgcccgaac ccgattaggt gtcgatattg |
| | gaaaaggtcc tgaggagcga |
| 661 | caattcaagg gtttaggtga ctgtattatg aaaatagcaa |
| | aatcagatgg aattgctggt |
| 721 | ttataccaag ggtttggtgt ttcagtacag ggcatcattg |
| | tgtaccgagc ctcttatttt |
| 781 | ggagcttatg acacagttaa gggtttatta ccaaagccaa |
| | agaaaactcc atttcttgtc |
| 841 | tcctttttca ttgctcaagt tgtgactaca tgctctggaa |
| | tactttctta tccctttgac |
| 901 | acagttagaa gacgtatgat gatgcagagt ggtgaggcta |
| | aacggcaata taaaggaacc |
| 961 | ttagactgct ttgtgaagat ataccaacat gaaggaatca |
| | gttccttttt tcgtggcgcc |
| 1021 | ttctccaatg ttcttcgcgg tacagggggt gctttggtgt |
| | tggtattata tgataaaatt |
| 1081 | aaagaattct ttcatattga tattggtggt aggtaatcgg |
| | gagagtaaat taagaaatac |
| 1141 | atggatttaa cttgttaaac atacaaatta catagctgcc |
| | atttgcatac attttgatag |
| 1201 | tgttattgtc tgtattttgt taaagtgcta gttctgcaat |
| | aaagcataca ttttttcaag |
| 1261 | aatttaaata ctaaaaatca gataaatgtg gattttcctc |
| | ccacttagac tcaaacacat |
| 1321 | tttagtgtga tatttcattt attataggta gtatatttta |
| | atttgttagt ttaaaattct |
| 1381 | ttttatgatt aaaaattaat catataatcc tagattaatg |
| | ctgaaatcta ggaaatgaaa |
| 1441 | gtagcgtctt ttaaattgct attcatttaa tatacctgtt |
| | ttcccatctt ttgaagtcat |
| 1501 | atggtatgac atatttctta aaagcttatc aatagatgtc |
| | atcatatgtg taggcagaaa |
| 1561 | taagctttgt tctatatctc ttctaagaca gttgttatta |
| | ctgtgtataa tatttacagt |
| 1621 | atcagccttt gattatagat gtgatcattt aaaatttgat |
| | aatgacttta gtgacattat |
| 1681 | aaaactgaaa ctggaaaata aaatggctta tctgctgatg |
| | tttatcttta aaataaataa |
| 1741 | aatcttgcta gtgtgaatat atcttagaac aaaaggtatc |
| | ctcttgaaaa ttagtttgta |
| 1801 | tattttgttg acaataaagg aagcttaact gttataaagg |
| | aaaaaaaaaa aaaaaa. |

In some embodiments of the disclosure, an ANT4 protein may comprise or consist of the amino acid sequence of ADP/APT translocase 4 (ANT4) (UniProtKB-Q9H0C2; also known as solute carrier family 25 member 31 (SLC25A31); transcript variant 2):

(SEQ ID NO: 3)

| | |
|---|---|
| 1 | MHREPAKKKA EKRLFDASSF GKDLLAGGVA AAVSKTAVAP |
| | IERVKLLLQV QASSKQISPE |
| 61 | ARYKGMVDCL VRIPREQGFF SFWRGNLANV IRYFPTQALN |
| | FAFKDKYKQL FMSGVNKEKQ |
| 121 | FWRWFLANLA SGGAAGATSL CVVYPLDFAR TRLGVDIGKG |
| | PEERQFKGLG DCIMKIAKSD |
| 181 | GIAGLYQGFG VSVQGIIVYR ASYFGAYDTV KGLLPKPKKT |
| | PFLVSFFIAQ VVTTCSGILS |
| 241 | YPFDTVRRRM MMQSGEAKRQ YKGTLDCFVK IYQHEGISSF |
| | FRGAFSNVLR GTGGALVLVL |
| 301 | YDKIKEFFHI DIGGR; |

or be encoded by the nucleic acid comprising or consisting of the sequence of:

(SEQ ID NO: 4)

| | |
|---|---|
| 1 | gcagcttttc cgcacgcgcc tcgccggcgc gcggctctct |
| | cagcgtccca agagccactt |
| 61 | tctcgccagt acgatgctgc agcggttttc cggttttccg |
| | cttcccttca tcgtagctcc |
| 121 | cgtactcatt tttagccact gctgccggtt tttatatcct |
| | tctccatcat gcatcgtgag |
| 181 | cctgcgaaaa agaaggcaga aaagcggctg tttgacgcct |
| | catccttcgg gaaggacctt |
| 241 | ctggccggcg gagtcgcggc agctgtgtcc aagacagcgg |
| | tggcgcccat cgagcgggtg |
| 301 | aagctgctgc tgcaggtgca ggcgtcgtcg aagcagatca |
| | gccccgaggc gcggtacaaa |
| 361 | ggcatggtgg actgcctggt gcggattcct cgcgagcagg |
| | gtttcttcag tttttggcgt |
| 421 | ggcaatttgg caaatgttat tcggtatttt ccaacacaag |
| | ctctaaactt tgcttttaag |
| 481 | gacaaataca agcagctatt catgtctgga gttaataaag |
| | aaaaacagtt ctggaggtgg |
| 541 | tttttggcaa acctggcttc tggtggagct gctggggcaa |
| | catccttatg tgtagtatat |
| 601 | cctctagatt ttgcccgaac ccgattaggt gtcgatattg |
| | gaaaaggtcc tgaggagcga |
| 661 | caattcaagg gtttaggtga ctgtattatg aaaatagcaa |
| | aatcagatgg aattgctggt |
| 721 | ttataccaag ggtttggtgt ttcagtacag ggcatcattg |
| | tgtaccgagc ctcttatttt |
| 781 | ggagcttatg acacagttaa gggtttatta ccaaagccaa |
| | agaaaactcc atttcttgtc |
| 841 | tcctttttca ttgctcaagt tgtgactaca tgctctggaa |
| | tactttctta tccctttgac |
| 901 | acagttagaa gacgtatgat gatgcaggtc attaatttcc |
| | ttataaatta caactcgaag |
| 961 | ctgcatctta aaaatttaga gtggtgaggc taaacggcaa |
| | tataaaggaa ccttagactg |
| 1021 | ctttgtgaag atataccaac atgaaggaat cagttccttt |
| | tttcgtggcg ccttctccaa |
| 1081 | tgttcttcgc ggtacagggg gtgctttggt gttggtatta |
| | tatgataaaa ttaaagaatt |
| 1141 | ctttcatatt gatattggtg gtaggtaatc gggagagtaa |
| | attaagaaat acatggattt |
| 1201 | aacttgttaa acatacaaat tacatagctg ccatttgcat |
| | acattttgat agtgttattg |
| 1261 | tctgtatttt gttaaagtgc tagttctgca ataaagcata |
| | cattttttca agaatttaaa |
| 1321 | tactaaaaat cagataaatg tggattttcc tcccacttag |
| | actcaaacac attttagtgt |
| 1381 | gatatttcat ttattatagg tagtatattt taatttgtta |
| | gtttaaaatt ctttttatga |
| 1441 | ttaaaaatta atcatataat cctagattaa tgctgaaatc |
| | taggaaatga aagtagcgtc |
| 1501 | ttttaaattg ctattcattt aatatacctg ttttcccatc |
| | ttttgaagtc atatggtatg |
| 1561 | acatatttct taaaagctta tcaatagatg tcatcatatg |
| | tgtaggcaga aataagcttt |
| 1621 | gttctatatc tcttctaaga cagttgttat tactgtgtat |
| | aatatttaca gtatcagcct |
| 1681 | ttgattatag atgtgatcat ttaaaatttg ataatgactt |
| | tagtgacatt ataaaactga |
| 1741 | aactggaaaa taaaatggct tatctgctga tgtttatctt |
| | taaaataaat aaaatcttgc |
| 1801 | tagtgtgaat atatcttaga acaaaaggta tcctcttgaa |
| | aattagtttg tatattttgt |
| 1861 | tgacaataaa ggaagcttaa ctgttataaa ggaaaaaaaa |
| | aaaaaaaa. |

We have identified a class of compounds, such as niclosamide ethanolamine (NEN) and BAM15 (N5,N6-bis(2-Fluorophenyl)-[1,2,5]oxadiazolo[3,4-b]pyrazine-5,6-diamine), that act as targeted mild mitochondrial uncouplers in sperm and can be utilized as sperm incapacitating agents. This rationale is that sperm mitochondrial uncoupling (sMU) drains sperm of energy and makes sperm unable to find and fertilize an egg. The search for specific activators of H⁺ leak via sperm-expressing ANT, is a novel strategy to develop nonhormonal contraceptives. Indeed, according to our data, NEN uncouples human sperm mitochondria, significantly decreases sperm beat frequency and hyperactivation. NEN is an oral salicylanilide derivative that has been approved by the US Food and Drug Administration (FDA) since 1958 for human use in the treatment of parasite tapeworm infections^{16,28}. It has been shown to act as a mitochondrial uncoupler by translocating protons across the inner mitochondrial membrane resulting in futile cycles of glucose and fatty acid oxidation^{2,15,31,32}. While its mitochondrial uncoupling is sufficient to kill gastrointestinal tapeworms, NEN has shown an excellent safety profile in humans²⁰, unlike DNP which is mildly toxic and has been associated with an unacceptably high rate of significant adverse effects²⁹. According to drug repurposing screening studies, niclosamide has strong in vivo and in vitro activity against antibiotic-resistant bacteria³³, and according to another study, may also inhibit Zika virus replication¹⁹. Moreover, niclosamide is recently characterized for uses in diabetes³², and human glioblastoma tumors³⁴, colon and ovarian cancers^{15,18,35}. While the exact molecular mechanism of niclosamide uncoupling action is not fully understood, similar to FA and DNP it should uncouple mitochondria by activating H⁺ leak via ANT, and according to our data it does so to sperm mitochondria. Therefore, our data, and the excellent safety profile of niclosamide, and its antimicrobial and antiviral properties indicate that NEN-based products exhibit contraceptive properties, in addition to their antimicrobial and antiviral properties.

Unless contraindicated or noted otherwise, in these descriptions and throughout this specification, the terms “a” and “an” mean one or more, the term “or” means and/or. The examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

### EXAMPLES

### Example 1: Identification of ANT as the Principal Mitochondrial Uncoupling Protein in Non-Fat Tissues

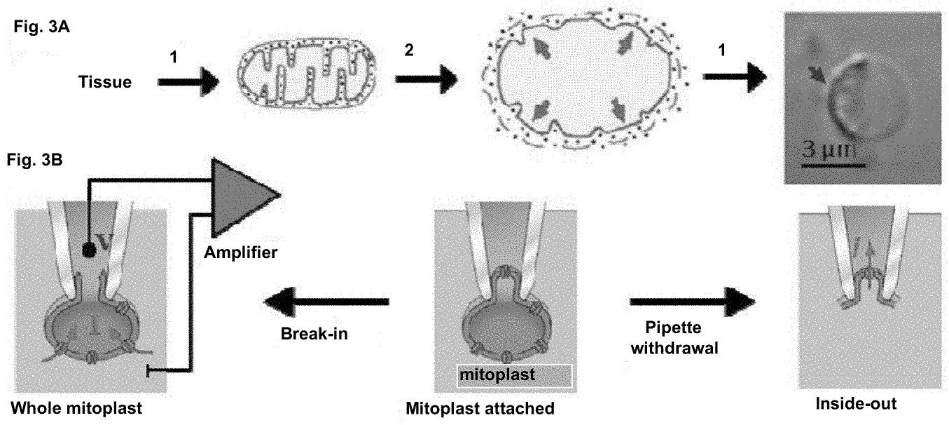

We employed direct patch-clamp recording from vesicles of the whole intact inner mitochondrial membrane (IMM; so-called mitoplasts, FIG. 3), which allow high-resolution functional analysis of mitochondrial ion channels and transporters in their native membrane environment^{10,36,37}. We recently used this method to characterize the mechanism by which fatty acids (FA) induce the H⁺ leak via uncoupling protein 1 (UCP1), the protein responsible formitochondrial thermogenesis in brown fat³⁷. However, UCP1 is fat-specific, and the mechanisms of thermogenesis in non-adipose tissues that constitute the majority of the body, are still poorly understood. Here we demonstrate that the whole-IMM patch clamp can be used to directly measure the UCP1-independent FA-induced H⁺ leak in non-adipose tissues

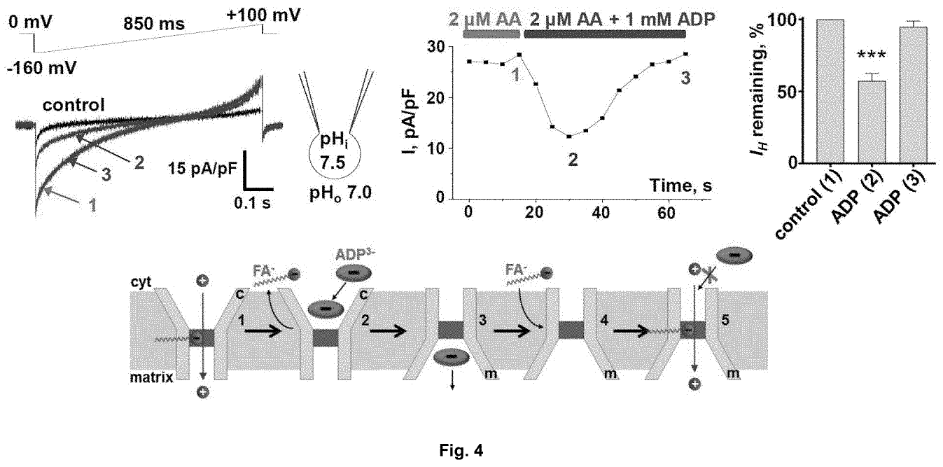

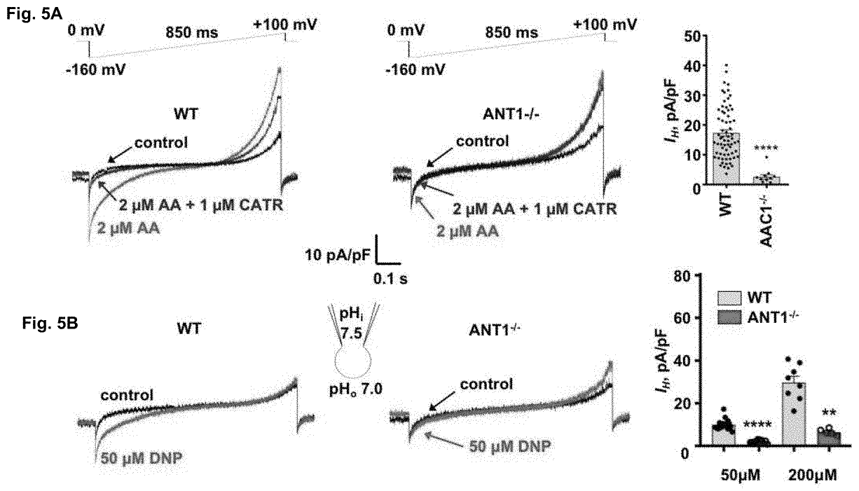

ANT is responsible for H⁺ leak across the IMM of non-adipose tissues and conducts H⁺ only in the presence of fatty acids (FA) (FIGS. 4 and 5)¹⁰. Additionally, using whole-IMM patch-clamp this group showed that mitochondrial uncoupler DNP strongly and specifically activates proton leak via ANT and can serve as pharmacological regulators of ANT-dependent mitochondrial uncoupling by mimicking ANT endogenous activator-FA. I_{H} mediated by ANT is blocked by a classical ANT inhibitor carboxyatractyloside (CATR, FIG. 5).

We recorded DNP-induced I_{H} in heart of ANT1-deficient mice (ANT1 is the dominant isoform of ANT in this tissue). These experiments demonstrate the I_{H} induced by DNP primarily depends on ANT (FIG. 5B-D). These results indicate that, in contrast to the previously proposed model, DNP does not uncouple mitochondria as simple chemical protonophores but target ANT. Thus, data demonstrate that the mitochondrial uncoupler-activated I_{H} can be recorded across the whole IMM using the patch-clamp method, and that this current is mediated by ANT.

### Example 2: Uncoupling of Human Sperm Mitochondria Reduces Sperm Motility and Prevents Fertilization

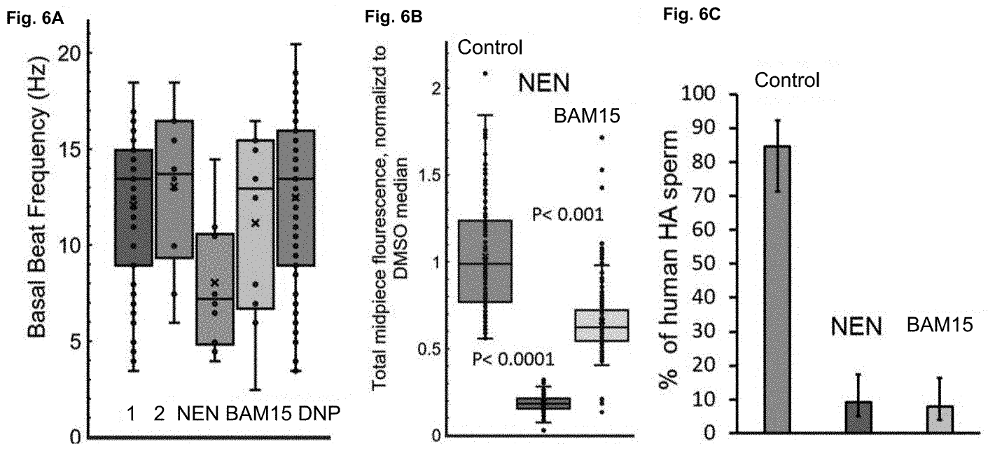

Here we have studied the effect of DNP, niclosamide, and BAM15, three known mitochondrial uncouplers, on their ability to uncouple sperm mitochondria^{13,38,39}. As shown in FIG. 6B, both BAM15 and NEN elicit strong sMU as assessed by mitochondrial depolarization. Importantly, niclosamide, the most potent of the two compounds, also decreased sperm beat frequency from 14 Hz to 6 Hz, (FIG. 6A), inhibited hyperactivatio (FIG. 6C) at concentrations as low as 1 μM.

### REFERENCES

1 Krauss, S., Zhang, C. Y. & Lowell, B. B. The mitochondrial uncoupling-protein homologues. Nat Rev Mol Cell Biol 6, 248-261, doi:10.1038/nrm1572 (2005).
2 Terada, H. Uncouplers of oxidative phosphorylation. Environmental health perspectives 87, 213-218, doi:10.1289/ehp.9087213 (1990).
3 Cannon, B. & Nedergaard, J. Brown adipose tissue: function and physiological significance. Physiol Rev 84, 277-359, doi:10.1152/physrev.00015.2003 (2004).
4 Lishko, P. V. et al. The control of male fertility by spermatozoan ion channels. Annu Rev Physiol 74, 453-475 (2012).
5 Wendler, A., Albrecht, C. & Wehling, M. Nongenomic actions of aldosterone and progesterone revisited. Steroids 77, 1002-1006, doi:10.1016/j.steroids.2011.12.023 (2012).
6 Daniels, K. & Mosher, W. D. Contraceptive methods women have ever used: United States, 1982-2010. National health statistics reports, 1-15 (2013).
7 Sedgh, G., Singh, S. & Hussain, R. Intended and unintended pregnancies worldwide in 2012 and recent trends. Studies in family planning 45, 301-314, doi:10.1111/j.1728-4465.2014.00393.x (2014).
8 Skovlund, C. W., Morch, L. S., Kessing, L. V. & Lidegaard, O. Association of Hormonal Contraception With Depression. JAMA psychiatry 73, 1154-1162, doi:10.1001/jamapsychiatry.2016.2387 (2016).
9 Skovlund, C. W., Morch, L. S., Kessing, L. V., Lange, T. & Lidegaard, O. Association of Hormonal Contraception With Suicide Attempts and Suicides. The American journal of psychiatry 175, 336-342, doi:10.1176/appi.ajp.2017. Ser. No. 17/060,616 (2018).
10 Bertholet, A. M. Chouchani, E. T.; Kazak, L.; Angelin, A.; Fedorenko, A.; Long, J. Z.; Vidoni, S.; Garrity, R.; Cho, J.; Terada, N.; Wallace, D. C.; Spiegelman, B. M.; and Kirichok, Y. Proton Transport is an Integral Function of the Mitochondrial ADP/ATP Carrier Nature July; 571(7766):515-520 (2019).
11 Mannowetz, N., Naidoo, N. M., Choo, S. A., Smith, J. F. & Lishko, P. V. Slol is the principal potassium channel of human spermatozoa. eLife 2, e01009, doi:10.7554/eLife.01009 (2013).
12 Azzu, V. & Brand, M. D. The on-off switches of the mitochondrial uncoupling proteins. Trends in biochemical sciences 35, 298-307, doi:10.1016/j.tibs.2009.11.001 (2010).
13 Brower, J. V., Lim, C. H., Jorgensen, M., Oh, S. P. & Terada, N. Adenine nucleotide translocase 4 deficiency leads to early meiotic arrest of murine male germ cells. Reproduction 138, 463-470, doi:10.1530/REP-09-0201 (2009).
14 Zhang, Y. et al. Human Adenine Nucleotide Translocase (ANT) Modulators Identified by High-Throughput Screening of Transgenic Yeast. Journal of biomolecular screening 21, 381-390, doi:10.1177/1087057115624637 (2016).
15 Kumar, R. et al. Mitochondrial uncoupling reveals a novel therapeutic opportunity for p53-defective cancers. Nat Commun 9, 3931, doi:10.1038/s41467-018-05805-1 (2018).
16 Al-Hadiya, B. M. Niclosamide: comprehensive profile. Profiles of drug substances, excipients, and related methodology 32, 67-96, doi:10.1016/50099-5428(05)32002-8 (2005).
17 World Health Organization, W. H. WHO Model List of Essential Medicines (19th List). (2015).
18 Walters Haygood, C. L. et al. Niclosamide Analogs for Treatment of Ovarian Cancer. International journal of gynecological cancer: official journal of the International Gynecological Cancer Society 25, 1377-1385, doi:10.1097/IGC.0000000000000506 (2015).
19 Xu, M. et al. Identification of small-molecule inhibitors of Zika virus infection and induced neural cell death via a drug repurposing screen. Nature medicine 22, 1101-1107, doi:10.1038/nm.4184 (2016).
20 U.S. National Library of Medicine (NLM), T. ToxNet: Niclosamide (Oral route). (2018).
21 Current Contraceptive Use in the United States, 2006-2010, and Changes in Patterns of Use Since 1995, <www.cdc.gov/nchs/data/nhsr060.pdf> (2012).
22 Mitchell, P. Coupling of phosphorylation to electron and hydrogen transfer by a chemiosmotic type of mechanism. Nature 191, 144-148 (1961).
23 Enerback, S. et al. Mice lacking mitochondrial uncoupling protein are cold-sensitive but not obese. Nature 387, 90-94, doi:10.1038/387090a0 (1997).
24 Shabalina, I. G. et al. UCP1 in brite/beige adipose tissue mitochondria is functionally thermogenic. Cell reports 5, 1196-1203, doi:10.1016/j.celrep.2013.10.044 (2013).
25 Cohen, P. & Spiegelman, B. M. Brown and Beige Fat: Molecular Parts of a Thermogenic Machine. Diabetes 64, 2346-2351, doi:10.2337/db15-0318 (2015).
26 Rolfe, D. F. & Brown, G. C. Cellular energy utilization and molecular origin of standard metabolic rate in mammals. Physiol Rev 77, 731-758, doi:10.1152/physrev.1997.77.3.731 (1997).
27 Parker, N., Affourtit, C., Vidal-Puig, A. & Brand, M. D. Energization-dependent endogenous activation of proton conductance in skeletal muscle mitochondria. Biochem J 412, 131-139, doi:10.1042/BJ20080006 (2008).
28 Weinbach, E. C. & Garbus, J. Mechanism of action of reagents that uncouple oxidative phosphorylation. Nature 221, 1016-1018 (1969).
29 Grundlingh, J., Dargan, P. I., El-Zanfaly, M. & Wood, D. M. 2,4-dinitrophenol (DNP): a weight loss agent with significant acute toxicity and risk of death. Journal of medical toxicology: official journal of the American College of Medical Toxicology 7, 205-212, doi:10.1007/s13181-011-0162-6 (2011).
30 McLaughlin, S. G. & Dilger, J. P. Transport of protons across membranes by weak acids. Physiol Rev 60, 825-863, doi:10.1152/physrev.1980.60.3.825 (1980).
31 Kadenbach, B. Intrinsic and extrinsic uncoupling of oxidative phosphorylation. Biochim Biophys Acta 1604, 77-94 (2003).
32 Tao, H., Zhang, Y., Zeng, X., Shulman, G. I. & Jin, S. Niclosamide ethanolamine-induced mild mitochondrial uncoupling improves diabetic symptoms in mice. Nature medicine 20, 1263-1269, doi:10.1038/nm.3699 (2014).
33 Rajamuthiah, R. et al. Repurposing salicylanilide anthelmintic drugs to combat drug resistant Staphylococcus aureus. PLoS One 10, e0124595, doi:10.1371/journal.pone.0124595 (2015).
34 Wieland, A. et al. Anticancer effects of niclosamide in human glioblastoma. Clinical cancer research: an official journal of the American Association for Cancer Research 19, 4124-4136, doi:10.1158/1078-0432.CCR-12-2895 (2013).
35 Alasadi, A. et al. Effect of mitochondrial uncouplers niclosamide ethanolamine (NEN) and oxyclozanide on hepatic metastasis of colon cancer. Cell death & disease 9, 215, doi:10.1038/s41419-017-0092-6 (2018).
36 Kirichok, Y., Krapivinsky, G. & Clapham, D. E. The mitochondrial calcium uniporter is a highly selective ion channel. Nature 427, 360-364, doi:10.1038/nature02246 (2004).
37 Fedorenko, A., Lishko, P. V. & Kirichok, Y. Mechanism of fatty-acid-dependent UCP1 uncoupling in brown fat mitochondria. Cell 151, 400-413, doi:10.1016/j.ce11.2012.09.010 (2012).
38 Rodic, N. et al. DNA methylation is required for silencing of ant4, an adenine nucleotide translocase selectively expressed in mouse embryonic stem cells and germ cells. Stem Cells 23, 1314-1323, doi:10.1634/stemcells.2005-0119 (2005).
39 Lim, C. H., Hamazaki, T., Braun, E. L., Wade, J. & Terada, N. Evolutionary genomics implies a specific function of Ant4 in mammalian and anole lizard male germ cells. PLoS One 6, e23122, doi:10.1371/journal.pone.0023122 (2011).

1. A method of promoting contraception, comprising administering to a person in need thereof a composition comprising an effective amount of a targeted mild mitochondria uncoupler.

2. The method of claim 1 wherein the uncoupler comprises a salicylanilide or salt thereof, such as an ethanolamine salt thereof, such as niclosamide ethanolamine (NEN).

3. The method of claim 1 wherein the uncoupler comprises a salicylanilide compound or salt thereof, and the salicylanilide compound is selected from: niclosamide, bromochlorosalicylanilide, oxyclozanide, rafoxanide, 3-tert-butyl-5-chloro-N-(2-chloro-4-nitrophenyl)-2-hydroxy-6-methylbenzamide, dibromsalan, metabromsalan, tribromsalan, and 2-iodo-N-phenylbenzamide (benodanil).

4. The method of claim 1 wherein the uncoupler comprises a salicylanilide compound or salt thereof, and the salicylanilide compound has a structure: 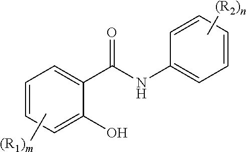
wherein R1 and R2 are independently halide, such as F, Cl, Br or I, substituted heteroatom selected from 0 and N, such as —OH, —NO2 or O-Ph-Cl, or lower (C1-C4) alkyl, such as Me, Et, or t-butyl, m is an integer 1, 2, 3 or 4, and n is an integer 1, 2, 3, 4 or 5.

5. The method of claim 1 wherein the uncoupler comprises BAM15 (N5,N6-bis(2-Fluorophenyl)-[1,2,5]oxadiazolo[3,4-b]pyrazine-5,6-diamine), or salt thereof.

6. The method of claim 1 wherein the composition is administered via an oral route, a topical route, a rectal route, or a vaginal route.

7. The method of claim 1, wherein the composition is administered as a pill, a cream, a vaginal ring, a vaginal film or a patch.

8. The method of claim 6, wherein the composition is administered orally and wherein the composition is administered as a pill.

9. The method of claim 6, wherein the composition is administered topically and wherein the composition is administered as a cream or a patch.

10. The method of claim 6 wherein the composition is administered intravaginally and wherein the composition is administered as a cream, a patch, a vaginal ring or a vaginal film.

11. The method of claim 6 wherein the composition is administered intrarectally and wherein the composition is administered as a cream or a patch.

12. Use of a targeted mild mitochondria uncoupler as a contraceptive.

13. The use of claim 12, wherein the uncoupler comprises a salicylanilide or salt thereof, such as an ethanolamine salt thereof, such as niclosamide ethanolamine (NEN).

14. The use of claim 12 wherein the uncoupler comprises a salicylanilide compound or salt thereof, and the salicylanilide compound is selected from: niclosamide, bromochlorosalicylanilide, oxyclozanide, rafoxanide, 3-tert-butyl-5-chloro-N-(2-chloro-4-nitrophenyl)-2-hydroxy-6-methylbenzamide, dibromsalan, metabromsalan, tribromsalan, and 2-iodo-N-phenylbenzamide (benodanil).

15. The use of claim 12 wherein the uncoupler comprises a salicylanilide compound or salt thereof, and the salicylanilide compound has a structure: 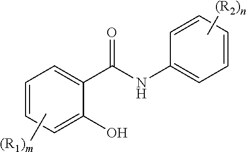
wherein R1 and R2 are independently halide, such as F, Cl, Br or I, substituted heteroatom selected from O and N, such as —OH, —NO2 or O-Ph-Cl, or lower (C1-C4) alkyl, such as Me, Et, or t-butyl, m is an integer 1, 2, 3 or 4, and n is an integer 1, 2, 3, 4 or 5.

16. The use of claim 12 wherein the uncoupler comprises BAM15 (N5,N6-bis(2-Fluorophenyl)-[1,2,5]oxadiazolo[3,4-b]pyrazine-5,6-diamine), or salt thereof.

17. The use of claim 12, wherein the composition is administered via an oral route, a topical route, a rectal route, or a vaginal route.

18. The use of claim 12, wherein the composition is administered as a pill, a cream, a vaginal ring, a vaginal film or a patch.

19. The use of claim 17, wherein the composition is administered orally and wherein the composition is administered as a pill; or the composition is administered topically and wherein the composition is administered as a cream or a patch; or the composition is administered intravaginally and wherein the composition is administered as a cream, a patch, a vaginal ring or a vaginal film; or the composition is administered intrarectally and wherein the composition is administered as a cream or a patch.

20. A contraceptive formulation comprising an effective amount of a targeted mild mitochondria uncoupler, preferably in the form of a pill, capsule, suppository, cream, vaginal ring, vaginal film or patch.


SEQUENCE LISTING (4 sequences)

SEQ ID NO: 1 - protein, 315 aa, human
MHREPAKKKAEKRLFDASSFGKDLLAGGVAAAVSKTAVAPIERVKLLLQVQASSKQISPEARYKGMVDCLVRIPREQGFFSFWRGNLANVIRYFPTQALNFAFKDKYKQLFMSGVNKEKQFWRWFLANLASGGAAGATSLCVVYPLDFARTRLGVDIGKGPEERQFKGLGDCIMKIAKSDGIAGLYQGFGVSVQGIIVYRASYFGAYDTVKGLLPKPKKTPFLVSFFIAQVVTTCSGILSYPFDTVRRRMMMQSGEAKRQYKGTLDCFVKIYQHEGISSFFRGAFSNVLRGTGGALVLVLYDKIKEFFHIDIGGR

SEQ ID NO: 2 - DNA, 1856 bp, human
gcagcttttccgcacgcgcctcgccggcgcgcggctctctcagcgtcccaagagccactttctcgccagtacgatgctgcagcggttttccggttttccgcttcccttcatcgtagctcccgtactcatttttagccactgctgccggtttttatatccttctccatcatgcatcgtgagcctgcgaaaaagaaggcagaaaagcggctgtttgacgcctcatccttcgggaaggaccttctggccggcggagtcgcggcagctgtgtccaagacagcggtggcgcccatcgagcgggtgaagctgctgctgcaggtgcaggcgtcgtcgaagcagatcagccccgaggcgcggtacaaaggcatggtggactgcctggtgcggattcctcgcgagcagggtttcttcagtttttggcgtggcaatttggcaaatgttattcggtattttccaacacaagctctaaactttgcttttaaggacaaatacaagcagctattcatgtctggagttaataaagaaaaacagttctggaggtggtttttggcaaacctggcttctggtggagctgctggggcaacatccttatgtgtagtatatcctctagattttgcccgaacccgattaggtgtcgatattggaaaaggtcctgaggagcgacaattcaagggtttaggtgactgtattatgaaaatagcaaaatcagatggaattgctggtttataccaagggtttggtgtttcagtacagggcatcattgtgtaccgagcctcttattttggagcttatgacacagttaagggtttattaccaaagccaaagaaaactccatttcttgtctcctttttcattgctcaagttgtgactacatgctctggaatactttcttatccctttgacacagttagaagacgtatgatgatgcagagtggtgaggctaaacggcaatataaaggaaccttagactgctttgtgaagatataccaacatgaaggaatcagttccttttttcgtggcgccttctccaatgttcttcgcggtacagggggtgctttggtgttggtattatatgataaaattaaagaattctttcatattgatattggtggtaggtaatcgggagagtaaattaagaaatacatggatttaacttgttaaacatacaaattacatagctgccatttgcatacattttgatagtgttattgtctgtattttgttaaagtgctagttctgcaataaagcatacattttttcaagaatttaaatactaaaaatcagataaatgtggattttcctcccacttagactcaaacacattttagtgtgatatttcatttattataggtagtatattttaatttgttagtttaaaattctttttatgattaaaaattaatcatataatcctagattaatgctgaaatctaggaaatgaaagtagcgtcttttaaattgctattcatttaatatacctgttttcccatcttttgaagtcatatggtatgacatatttcttaaaagcttatcaatagatgtcatcatatgtgtaggcagaaataagctttgttctatatctcttctaagacagttgttattactgtgtataatatttacagtatcagcctttgattatagatgtgatcatttaaaatttgataatgactttagtgacattataaaactgaaactggaaaataaaatggcttatctgctgatgtttatctttaaaataaataaaatcttgctagtgtgaatatatcttagaacaaaaggtatcctcttgaaaattagtttgtatattttgttgacaataaaggaagcttaactgttataaaggaaaaaaaaaaaaaaaa

SEQ ID NO: 3 - protein, 315 aa, human; identical to SEQ ID NO: 1

SEQ ID NO: 4 - DNA, 1908 bp, human
gcagcttttccgcacgcgcctcgccggcgcgcggctctctcagcgtcccaagagccactttctcgccagtacgatgctgcagcggttttccggttttccgcttcccttcatcgtagctcccgtactcatttttagccactgctgccggtttttatatccttctccatcatgcatcgtgagcctgcgaaaaagaaggcagaaaagcggctgtttgacgcctcatccttcgggaaggaccttctggccggcggagtcgcggcagctgtgtccaagacagcggtggcgcccatcgagcgggtgaagctgctgctgcaggtgcaggcgtcgtcgaagcagatcagccccgaggcgcggtacaaaggcatggtggactgcctggtgcggattcctcgcgagcagggtttcttcagtttttggcgtggcaatttggcaaatgttattcggtattttccaacacaagctctaaactttgcttttaaggacaaatacaagcagctattcatgtctggagttaataaagaaaaacagttctggaggtggtttttggcaaacctggcttctggtggagctgctggggcaacatccttatgtgtagtatatcctctagattttgcccgaacccgattaggtgtcgatattggaaaaggtcctgaggagcgacaattcaagggtttaggtgactgtattatgaaaatagcaaaatcagatggaattgctggtttataccaagggtttggtgtttcagtacagggcatcattgtgtaccgagcctcttattttggagcttatgacacagttaagggtttattaccaaagccaaagaaaactccatttcttgtctcctttttcattgctcaagttgtgactacatgctctggaatactttcttatccctttgacacagttagaagacgtatgatgatgcaggtcattaatttccttataaattacaactcgaagctgcatcttaaaaatttagagtggtgaggctaaacggcaatataaaggaaccttagactgctttgtgaagatataccaacatgaaggaatcagttccttttttcgtggcgccttctccaatgttcttcgcggtacagggggtgctttggtgttggtattatatgataaaattaaagaattctttcatattgatattggtggtaggtaatcgggagagtaaattaagaaatacatggatttaacttgttaaacatacaaattacatagctgccatttgcatacattttgatagtgttattgtctgtattttgttaaagtgctagttctgcaataaagcatacattttttcaagaatttaaatactaaaaatcagataaatgtggattttcctcccacttagactcaaacacattttagtgtgatatttcatttattataggtagtatattttaatttgttagtttaaaattctttttatgattaaaaattaatcatataatcctagattaatgctgaaatctaggaaatgaaagtagcgtcttttaaattgctattcatttaatatacctgttttcccatcttttgaagtcatatggtatgacatatttcttaaaagcttatcaatagatgtcatcatatgtgtaggcagaaataagctttgttctatatctcttctaagacagttgttattactgtgtataatatttacagtatcagcctttgattatagatgtgatcatttaaaatttgataatgactttagtgacattataaaactgaaactggaaaataaaatggcttatctgctgatgtttatctttaaaataaataaaatcttgctagtgtgaatatatcttagaacaaaaggtatcctcttgaaaattagtttgtatattttgttgacaataaaggaagcttaactgttataaaggaaaaaaaaaaaaaaaa